(19) United States
(12) Patent Application Publication
(10) Pub. No.: US 2022/0099624 A1
(43) Pub. Date: Mar. 31, 2022

(54) NUCLEIC ACID DETECTION HOST AND NUCLEIC ACID DETECTION DEVICE

(71) Applicant: iCare Diagnostics International Co. Ltd., New Taipei City (TW)

(72) Inventors: CHIA-HSIN CHANG, New Taipei (TW); PO-CHING HUANG, New Taipei (TW); PENG-YU CHIU, New Taipei (TW); CHIEH-CHUNG CHUNG, New Taipei (TW); CHUN-CHIH CHEN, New Taipei (TW); HSIN-WEI WANG, New Taipei (TW)

(21) Appl. No.: 17/488,672

(22) Filed: Sep. 29, 2021

Related U.S. Application Data
(60) Provisional application No. 63/085,368, filed on Sep. 30, 2020.
(60) Provisional application No. 63/085,385, filed on Sep. 30, 2020.

(30) Foreign Application Priority Data
Jun. 30, 2021 (CN) 202110735243.X

(51) Int. Cl. G01N 27/447; C12Q 1/686; B01L 7/00

(52) U.S. Cl. CPC G01N 27/44721; C12Q 1/686; B01L 2300/1805; G01N 27/44756; G01N 27/4473; B01L 7/52

(57) ABSTRACT

A nucleic acid detection host includes a host body, a detection kit installation area, a sample heating area, a sampling groove, a heating structure, and an image collection unit. The detection kit installation area is configured to detachably install a nucleic acid detection kit. The sampling groove is disposed on the detection kit installation area and is connected to the detection kit installation area. The heating structure is configured to heat the nucleic acid detection kit to perform a PCR amplification reaction and an electrophoretic detection. The image collection unit is configured to collect an image of the nucleic acid detection kit. A nucleic acid detection device including the nucleic acid detection host is also disclosed. The nucleic acid detection device has a simple structure, which is portable, flexible, and convenient, and can be used at home.

### FIELD

The subject matter relates to nucleic acid detection, and more particularly, to a nucleic acid detection host and a nucleic acid detection device with the nucleic acid detection host.

### BACKGROUND

At present, molecular diagnosis, morphological detection, and immunological detection are mostly carried out in laboratories. Detection processes are time-consuming, inefficient, and inflexible, and detection devices are generally not portable. Therefore, detection cannot be carried out anytime and anywhere. Especially, patients with highly infectious virus may infect others on the way to the laboratories, which has potential safety hazards.

### BRIEF DESCRIPTION OF THE DRAWINGS

Implementations of the present technology will now be described, by way of example only, with reference to the attached figures.

FIG. 1 is a diagrammatic view of an embodiment of a nucleic acid detection device according to the present disclosure.

FIG. 2 is a diagrammatic view of an embodiment of a nucleic acid detection host according to the present disclosure.

FIG. 3 is a cross-sectional view taken along III-III of FIG. 2.

FIG. 4 is a cross-sectional view taken along IV-IV of FIG. 1.

FIG. 5 is an enlarged view of a portion of the nucleic acid detection device labeled A in FIG. 4.

FIG. 6 is a cross-sectional view taken along VI-VI of FIG. 2.

FIG. 7 is an explosion diagrammatic view of the nucleic acid detection host according to the present disclosure.

FIG. 8 is a front diagrammatic view of an embodiment of a nucleic acid detection kit according to the present disclosure.

FIG. 9 is a rear diagrammatic view of the nucleic acid detection kit according to the present disclosure.

FIG. 10 is an explosion diagrammatic view of an embodiment of a liquid transfer unit according to the present disclosure.

FIG. 11 is a diagrammatic view of the liquid transfer unit according to the present disclosure.

FIG. 12 is a diagrammatic view of an embodiment of a collection cup and a reagent package according to the present disclosure.

FIG. 13 to FIG. 17 are diagrammatic views showing steps of a nucleic acid detecting process according to the present disclosure.

FIG. 18 is a fluorescent image of a nucleic acid detection result according to the present disclosure.

FIG. 19 is a diagrammatic view of another embodiment of a nucleic acid detection host according to the present disclosure.

FIG. 20 is a diagrammatic view of another embodiment of a nucleic acid detection host with a pressing assembly according to the present disclosure.

### DETAILED DESCRIPTION

It will be appreciated that for simplicity and clarity of illustration, where appropriate, reference numerals have been repeated among the different figures to indicate corresponding or analogous components. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure.

The term “comprising,” when utilized, means “including, but not necessarily limited to”; it specifically indicates open-ended inclusion or membership in the so-described combination, group, series, and the like.

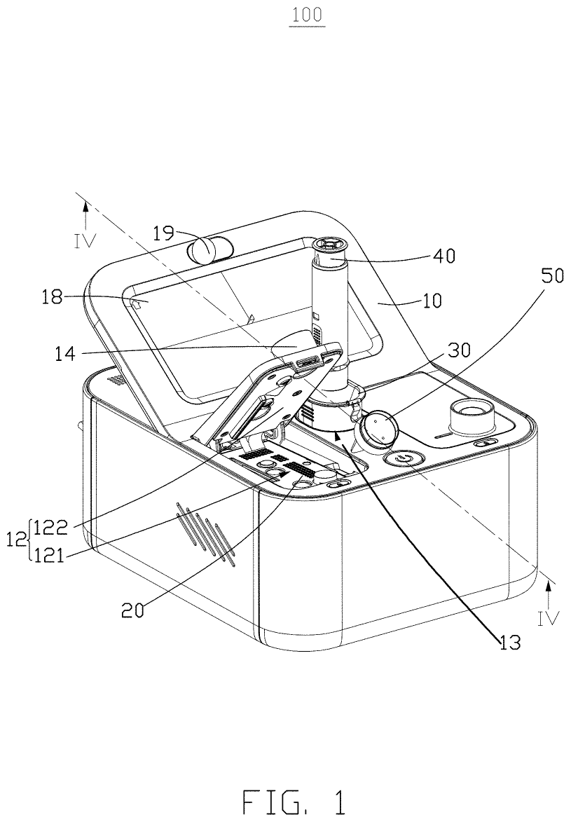

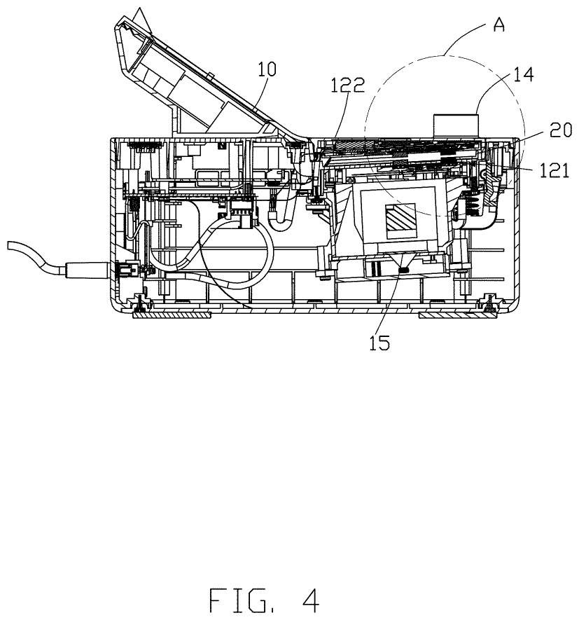

FIGS. 1-4 illustrate a nucleic acid detection device 100 according to the present disclosure. The nucleic acid detection device 100 includes a nucleic acid detection host 10, a nucleic acid detection kit 20, a collection cup 30, and a liquid transfer unit 40. The nucleic acid detection host 10 includes a host body 11, a detection kit installation area 12, a sample heating area 13, a sampling groove 14, a heating structure 17, an image collection unit 15, and a controller 16. The detection kit installation area 12, the sample heating area 13, the sampling groove 14, the heating structure 17, the image collection unit 15, and the controller 16 are disposed on the host body 11. The detection kit installation area 12, the sample heating area 13, the heating structure 17, and the image collection unit 15 are electrically connected to the controller 16. The heating structure 17 is disposed in the detection kit installation area 12. The nucleic acid detection kit 20 is detachably disposed in the detection kit installation area 12. The collection cup 30 is detachably disposed in the sample heating area 13. The liquid transfer unit 40 is detachably connected to the collection cup 30, and detachably disposed in the sampling groove 14. The collection cup 30 is used to receive a detection solution. The liquid transfer unit 40 is used to quantitatively absorb the detection solution from the collection cup 30, and to add the detection solution into the nucleic acid detection kit 20 through the sampling groove 14. The nucleic acid detection kit 20 is configured to perform a PCR amplification reaction and an electrophoretic detection successively.

Referring to FIGS. 1-4, the detection kit installation area 12 includes a mounting groove 121 and a cover plate 122 detachably disposed on the mounting groove 121. The nucleic acid detection kit 20 is manually inserted into the mounting groove 121. The nucleic acid detection kit 20 is electrically connected to the controller 16 and the heating structure 17. The cover plate 122 is configured to close and open the mounting groove 121 to facilitate the insertion and removal of the nucleic acid detection kit 20. The sample heating area 13 is configured to collect a nucleic acid sample of an object. The nucleic acid sample is mixed with a detection agent (such as a buffer solution) in the sample heating area 13 to form a detection solution. The sample heating area 13 is further configured to heat the detection solution under the control of the controller 16. The sampling groove 14 is disposed on the cover plate 122 and is connected to the detection kit installation area 12. The sampling groove 14 is disposed to add the detection solution into the nucleic acid detection kit 20 in the mounting groove 121. The heating structure 17 is disposed to heat the nucleic acid detection kit 20 to perform the PCR amplification reaction and the electrophoretic detection. The image collection unit 15 is disposed on a side of the mounting groove 121 away from the sampling groove 14. The image collection unit 15 is configured to collect an image of the nucleic acid detection kit 20 in the detection kit installation area 12 under the control of the controller 16. The image is a fluorescent image of the electrophoretic detection, and a result can be obtained according to the fluorescent image.

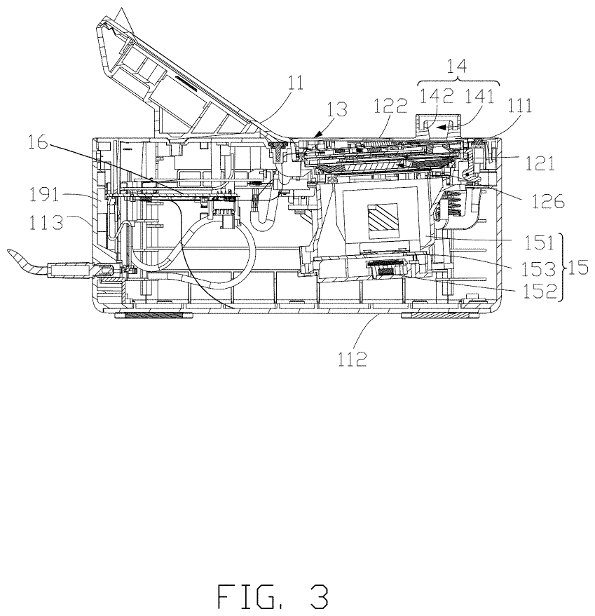

Referring to FIG. 3, the host body 11 includes a first surface 111, a second surface 112 opposite the first surface 111, and a sidewall 113 connecting the first surface 111 and the second surface 112. The first surface 111 defines an opening of the detection kit installation area 12, and the nucleic acid detection kit 20 can be placed in the detection kit installation area 12 through the opening. the first surface 111 further defines an opening of the sampling groove 14 and an opening of the sample heating area 13.

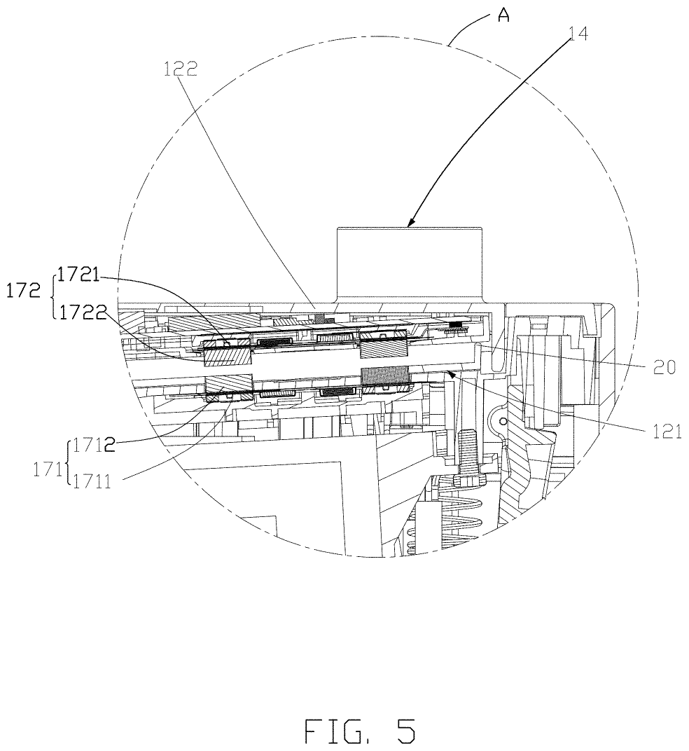

Referring to FIGS. 3 and 5, the mounting groove 121 is inclined relative to the first surface 111, which can make the nucleic acid detection kit 20 be placed obliquely in the mounting groove 121. In an embodiment, relative to the first surface 111, a height of an end of the mounting groove 121 closed to the sampling groove 14 is lower than a height of another end of the mounting groove 121 away from the sampling groove 14. Bubbles will be generated during the PCR amplification reaction in the nucleic acid detection kit 20. The bubbles may stay in and block a flow path of microbeads in the nucleic acid detection kit 20, so that the microbeads cannot move in the flow path, which causes a failure of the PCR amplification reaction. Therefore, the mounting groove 121 is designed to be inclined, so that the nucleic acid detection kit 20 can be placed obliquely, and the bubbles generated by the PCR amplification reaction can be discharged out naturally without hindering the movement of the microbeads.

A shape of the mounting groove 121 may be designed according to a shape of the nucleic acid detection kit 20. In an embodiment, the mounting groove 121 is substantially rectangular.

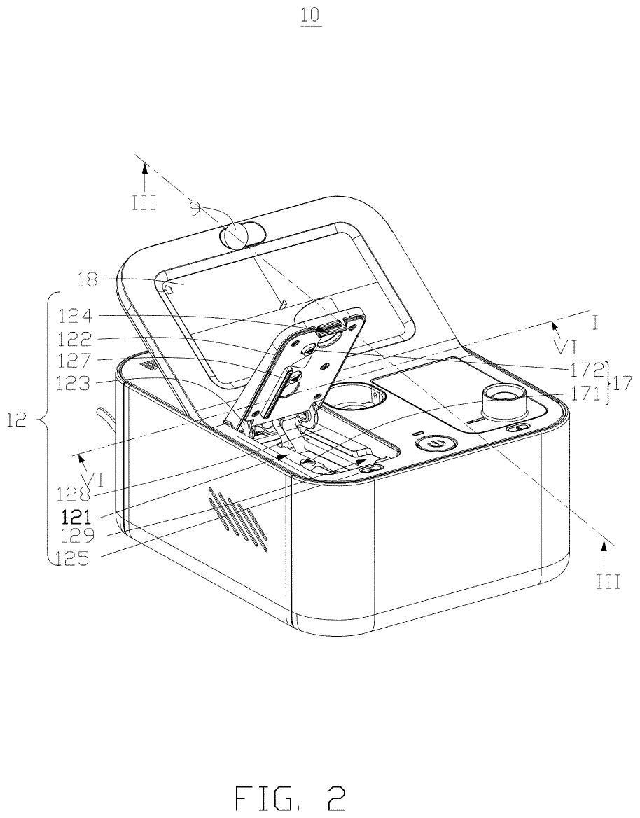

In an embodiment, referring to FIGS. 1 and 2, the cover plate 122 is disposed to close or open the mounting groove 121. The sampling groove 14 is disposed at one end of the cover plate 122, the other end of the cover plate 122 away from the sampling groove 14 includes a rotating connector 123. The cover plate 122 is rotatably disposed on a sidewall of the mounting groove 121 through the rotating connector 123. The cover plate 122 can rotate to open and close the mounting groove 121.

In an embodiment, a sidewall of the one end of the cover plate 122 close to the sampling groove 14 defines a slot 124. A clamping block 125 corresponding to the slot 124 is disposed in the mounting groove 121. The clamping block 125 can be clamped into the slot 124 to fix the cover plate 122 in an opening of the mounting groove 121, so as to avoid accidental opening of the cover plate 122. The cover plate 122 can be opened by pressing the clamping block 125 to withdraw from the slot 124.

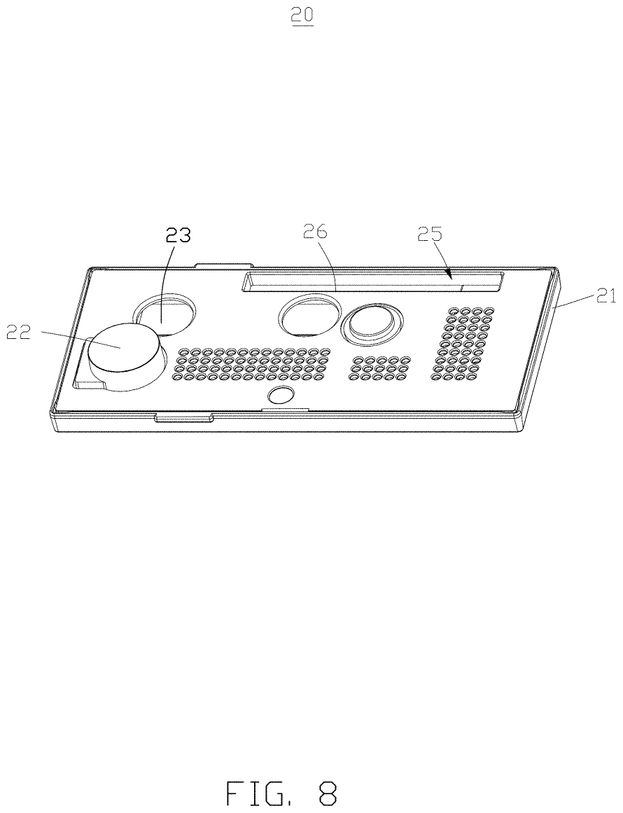

Referring to FIGS. 1-3 and 8, the detection kit installation area 12 further includes an imaging port 126 disposed on a bottom surface of the mounting groove 121, a host connector 127 disposed on a surface of the cover plate 122 close to the mounting groove 121, and a first sensor (not shown in the figures) disposed in the mounting groove 121. The first sensor is connected to the controller 16. The image collection unit 15 can collect the fluorescent image in the nucleic acid detection kit 20 through the imaging port 126. After the cover plate 122 covers the mounting groove 121, the host connector 127 is electrically connected to a detection kit connector 26 of the nucleic acid detection kit 20, thereby controlling the nucleic acid detection kit 20 to perform the PCR amplification reaction and the electrophoretic detection. The first sensor senses whether the nucleic acid detection kit 20 is inserted into the mounting groove 121, and transmits a signal to the controller 16 to control the nucleic acid detection kit 20 to start the nucleic acid detection.

In an embodiment, the host connector 127 is strip-shaped. The nucleic acid detection kit 20 defines a card slot 25. The detection kit connector 26 is disposed in the card slot 25. When the cover plate 122 covers the mounting groove 121, the host connector 127 can be inserted into the card slot 25 and then is electrically connected to the detection kit connector 26. The nucleic acid detection kit 20 is also fixed by the connection of the host connector 127 and the card slot 25 to prevent the nucleic acid detection kit 20 from moving in the mounting groove 121.

In an embodiment, the mounting groove 121 is provided with two fixing blocks 128, which are respectively disposed on two opposite sides of the imaging port 126. The two fixing blocks 128 are used to clamp the nucleic acid detection kit 20 and prevent the nucleic acid detection kit 20 from moving in the mounting groove 121. Each of the two fixing blocks 128 defines an avoidance groove 129, which can facilitate the insertion and removal of the nucleic acid detection kit 20.

In an embodiment, a distance between the two fixing blocks 128 is slightly greater than a width of the nucleic acid detection kit 20. Thus, the nucleic acid detection kit 20 can be stably fixed in the mounting groove 121.

In an embodiment, the cover plate 122 defines a through hole (not shown in the figures), and the through hole corresponds to the sampling groove 14. The sampling groove 14 is connected to the mounting groove 121 through the through hole.

In an embodiment, the first sensor is used to sense whether the nucleic acid detection kit 20 is inserted into or ejected from the mounting groove 121, and the sensing result is used to initiate or end the nucleic acid detection. When the first sensor detects that the nucleic acid detection kit 20 is inserted into the mounting groove 121, the nucleic acid detection is started. When the first sensor detects that the nucleic acid detection kit 20 is ejected from the mounting groove 121, the nucleic acid detection is ended.

In an embodiment, referring to FIG. 3, the imaging port 126 is substantially rectangular. The imaging port 126 should make sure that the image collection unit 15 can collect a complete fluorescent image in the nucleic acid detection kit 20 through the imaging port 126.

In an embodiment, the image collection unit 15 is disposed directly below the imaging port 126.

Referring to FIG. 1, the number of the detection kit installation areas 12 is two. The two detection kit installation areas 12 are located on two opposite sides of the sample heating area 13. The two detection kit installation areas 12 can carry out two groups of the PCR amplification reaction and the electrophoretic detection at the same time, thereby improving detection efficiency and space utilization of the nucleic acid detection device 100.

In an embodiment, an isolation layer (not shown) is set between the two detection kit installation areas 12 to avoid temperature interference between the two detection kit installation areas 12, when the two groups of the PCR amplification reactions are carried out at the same time. The isolation layer can effectively isolate the two detection kit installation areas 12 to avoid the temperature interference and improve the temperature accuracy of the PCR amplification reactions.

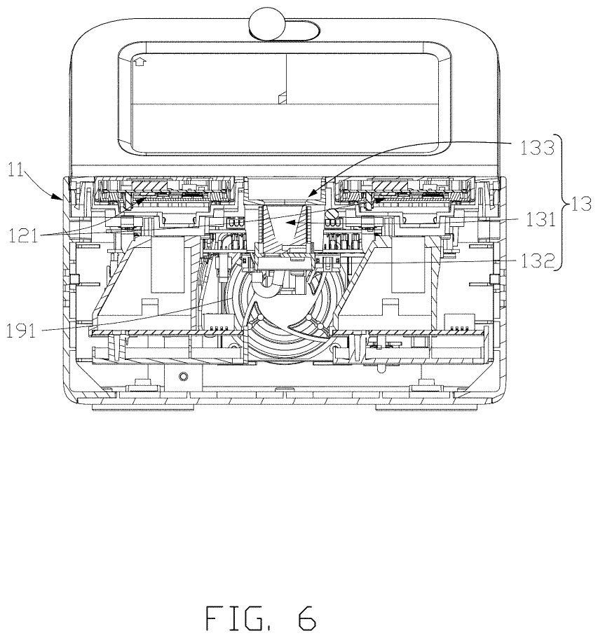

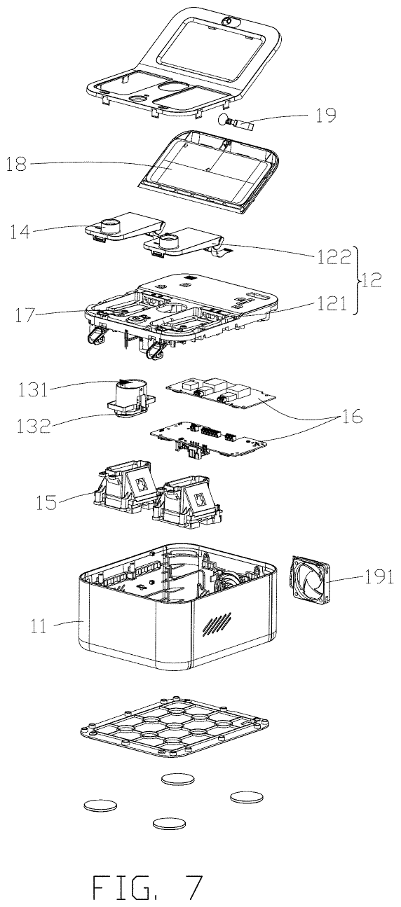

Referring to FIGS. 6 and 7, the sample heating area 13 includes a holding tank 131 and a heating block 132 disposed at the bottom of the holding tank 131. The heating block 132 is electrically connected to the controller 16. The controller 16 can energize the heating block 132 to heat the heating block 132. The controller 16 can also detect a temperature and a heating time of the heating block 132 through a temperature sensor (not shown in the figures) and a time relay (not shown in the figures), respectively. In an embodiment, the heating temperature of the heating block 132 is about 95° C., and the heating time is about 5 minutes. After heating, the heating block 132 is cooled at room temperature or at a certain temperature (such as below 40° C.).

In an embodiment, the heating block 132 is an aluminum block, a copper block, or other heating structures such as heating wire, heating coating, and heating sheet. At the same time, the sample heating area 13 is also provided with a second sensor (not shown in the figure). The second sensor is electrically connected to the controller 16. Referring to FIGS. 1 and 6, whether the collection cup 30 is inserted into the holding tank 131 can be sensed by the second sensor. When the collection cup 30 is inserted into the holding tank 131, the second sensor sends a trigger signal to the controller 16 to start the heating process.

In an embodiment, an opening of the holding tank 131 is disposed on the first surface 111, and the opening of the holding tank 131 is substantially elliptical. A shape of the holding tank 131 can be specifically designed according to a shape of the collection cup 30.

In an embodiment, referring to FIG. 6, the holding tank 131 is provided with a first clamping position 133 for clamping the collection cup 30.

Referring to FIG. 3, the bottom of the sampling groove 14 defines a sampling channel 141. the first surface 111 further defines an opening of the sampling groove 14. The sampling channel 141 is inserted into the mounting groove 121. An end of the sampling channel 141 close to the mounting groove 121 is connected to the nucleic acid detection kit 20. The detection solution enters the nucleic acid detection kit 20 in the mounting groove 121 through the sampling channel 141.

In an embodiment, the sampling channel 141 is funnel-shaped.

In an embodiment, the sampling groove 14 is further provided with a second clamping position 142 for clamping the liquid transfer unit 40.

Referring to FIGS. 1, 2, 5, and 8, the heating structure 17 includes a first heating component 171 disposed in the mounting groove 121 and a second heating component 172 disposed on the surface of the cover plate 122 close to the mounting groove 121. The first heating component 171 and the second heating component 172 are electrically connected to the controller 16. By setting two groups of heating components, upper and lower surfaces of the nucleic acid detection kit 20 can be heated at the same time, so that the detection solution can be heated evenly, and the PCR amplification reaction can be more sufficient. In addition, the heating structure 17 is disposed on the nucleic acid detection host 10 instead on the nucleic acid detection kit 20, which can reduce a cost of the nucleic acid detection kit 20.

In an embodiment, the first heating component 171 includes a first circuit board 1711 and a plurality of first heaters 1712 disposed on the first circuit board 1711. Each first heater 1712 corresponds to a corresponding one of the PCR amplification reaction areas of the nucleic acid detection kit 20. The first circuit board 1711 is disposed on a side of the mounting groove 121 away from the cover plate 122. The first heater 1712 extends in the mounting groove 121 and connects to the lower surface of the nucleic acid detection kit 20 in the mounting groove 121.

In an embodiment, the second heating component 172 includes a second circuit board 1721 and a plurality of second heaters 1722 disposed on the second circuit board 1721. Each second heater 1722 corresponds to a corresponding one of the PCR amplification reaction areas of the nucleic acid detection kit 20. The second circuit board 1721 is disposed inside the cover plate 122. The second heater 1722 protrudes from a surface of the cover plate 122 close to the mounting groove 121 to connect to the upper surface of the nucleic acid detection kit 20 in the mounting groove 121.

In an embodiment, the number of the first heaters 1712 can be two. One of the two first heaters 1712 has a heating temperature range of 40° C.-75° C., and other one of the two first heaters 1712 has a heating temperature range of 90° C.-105° C.

In an embodiment, the number of the second heaters 1722 can be two. One of the two second heaters 1722 has a heating temperature range of 40° C.-75° C., and other one of the two second heaters 1722 has a heating temperature range of 90° C.-105° C.

In an embodiment, the first heaters 1712 and the second heaters 1722 are aluminum blocks, copper blocks, or other heating structures such as heating wire, heating coating, and heating sheet.

In yet another embodiment, the number of the first heaters 1712 can be three. One of the three first heaters 1712 has a heating temperature range of 40° C.-65° C., the second of the three first heaters 1712 has a heating temperature range of 68° C.-75° C., and the third of the three first heaters 1712 has a heating temperature range of 90° C.-105° C.

In yet another embodiment, the number of the second heaters 1722 can be three. One of the three second heaters 1722 has a heating temperature range of 40° C.-65° C., the second of the three second heaters 1722 has a heating temperature range of 68° C.-75° C., and the third of the three second heaters 1722 has a heating temperature range of 90° C.-105° C.

Referring to FIGS. 2-4, the image collection unit 15 includes a fixing box 151 disposed on a side of the mounting groove 121 away from the sampling groove 14, a light source (not shown) disposed in the fixing box 151, an image collection control board 153, and an image collector 152. The light source and the image collector 152 are electrically connected to the image collection control board 153. The image collection control board 153 is electrically connected to the controller 16. The light source is used to emit light to the imaging port 126 under the control of the image collection control board 153. The image collector 152 is used to collect fluorescent images in the nucleic acid detection kit 20 under the control of the controller 16.

In an embodiment, the fixing box 151 has an inverted conical funnel structure. A sidewall of the fixing box 151 is inclined with respect to the first surface 111. A size of an end of the fixing box 151 close to the first surface 111 is smaller than a size of another end of the fixing box 151 away from the first surface 111. The end of the fixing box 151 close to the first surface 111 is connected to the mounting groove 121 through the imaging port 126. The image collector 152 and the light source correspond to the imaging port 126. The inclined sidewall of the fixing box 151 can focus and concentrate a light passing through the imaging port 126 during imaging.

In an embodiment, the inner surface of the sidewall of the fixing box 151 is covered by a reflective coating, which can reflect the light into the imaging port 126.

In an embodiment, the controller 16 includes an image processor (not shown in the figures). The fluorescent image collected by the image collector 152 is transmitted to the image processor for processing, and a processed image is further output.

In an embodiment, the controller 16 further includes a memory (not shown) for storing detection results and information related to the detection process.

Referring to FIGS. 1 and 2, the nucleic acid detection host 10 further includes a display screen 18 and a camera 19, which are electrically connected to the controller 16. The display screen 18 is disposed to display an operation interface to allow a user to set operation parameters, and also disposed to display detection images. The camera 19 is disposed to record an operation process of the user, and collect relevant information of the detection solution (such as information indicating a source of the nucleic acid sample).

Referring to FIG. 3, the nucleic acid detection host 10 further includes a heat dissipation structure 191, which is electrically connected to the controller 16 to dissipate heat for the nucleic acid detection host 10.

In an embodiment, the heat dissipation structure 191 is a heat dissipation fan. The heat dissipation structure 191 is disposed on the sidewall 113. A plurality of heat dissipation vents is disposed on the host body 11 to discharge the heat inside the nucleic acid detection host 10.

Cooperation between the nucleic acid detection host 10 and the nucleic acid detection kit 20 in the nucleic acid detection device 100 can perform the PCR amplification reaction and the electrophoretic detection in one piece of device. The fluorescent image displayed on the display screen 18 is the image of the electrophoretic detection. The nucleic acid detection device 100 integrates the heating, sampling, detecting, and result outputting into a single equipment. Thus, the nucleic acid detection device 100 has a simple structure, which is portable, flexible, and convenient, and can be used at home.

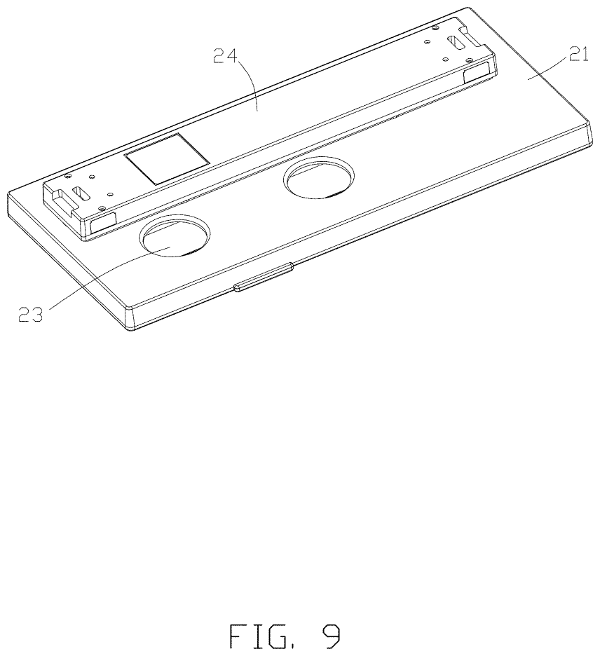

Referring to FIGS. 1, 3, 8, and 9, the nucleic acid detection kit 20 integrates the PCR amplification reaction process with the electrophoretic detection process, and the detection solution directly enters the electrophoretic box for the electrophoretic detection after the PCR amplification reaction. The nucleic acid detection kit 20 includes a kit body 21, a sampling port 22 disposed on the kit body 21, a detection chip 23, an electrophoretic box 24, and the detection kit connector 26. The sampling port 22 close to the sampling groove 14. An end of the sampling channel 141 away firm the sampling groove 14 is connected to the sampling port 22. The sampling port 22 is used to add the detection solution into the detection chip 23. The detection chip 23 is disposed in the kit body 21. The electrophoretic box 24 is disposed outside the kit body 21. The kit body 21 is connected to the electrophoretic box 24. The detection chip 23 is used to perform the PCR amplification reaction. The electrophoretic box 24 is used to perform the electrophoretic detection. The imaging port 126 corresponds to the electrophoretic box 24. The image collection unit 15 acquires the fluorescent images of the electrophoretic box 24 through the imaging port 126. The detection kit connector 26 is disposed in the card slot 25, and the host connector 127 in the detection kit installation area 12 can be clamped into the card slot 25 and electrically connected to the detection kit connector 26. The detection kit connector 26 is electrically connected to the detection chip 23 and the electrophoretic box 24. When the PCR amplification reaction is completed, the electrophoretic detection can be carried out automatically. The two processes are performed in a single equipment, and the sampling accuracy is controlled accurately. The nucleic acid detection kit 20 integrates with the detection chip 23 and the electrophoretic box 24, which has a small size, and is suitable for the nucleic acid detection device 100.

In an embodiment, the nucleic acid detection kit 20 is disposable. The nucleic acid detection kit 20 has no need to be cleaned after used.

In an embodiment, the nucleic acid detection kit 20 has substantially a cubic structure.

Referring to FIGS. 1 and 10-12, the collection cup 30 and the liquid transfer unit 40 can be clamped together. The collection cup 30 is used to collect the nucleic acid sample (such as saliva or other liquid sample), which is mixed with a detection reagent to form the detection solution. The detection solution is then heated in the sample heating area 13. The liquid transfer unit 40 is used to quantitatively absorb the detection solution from the collection cup 30, and add the detection solution into the nucleic acid detection kit 20 through the sampling groove 14.

In an embodiment, a conical groove is disposed inside the collection cup 30. After spitting saliva into the collection cup 30, the saliva can be concentrated in the bottom of the conical groove to facilitate the collection of a small amount of nucleic acid sample.

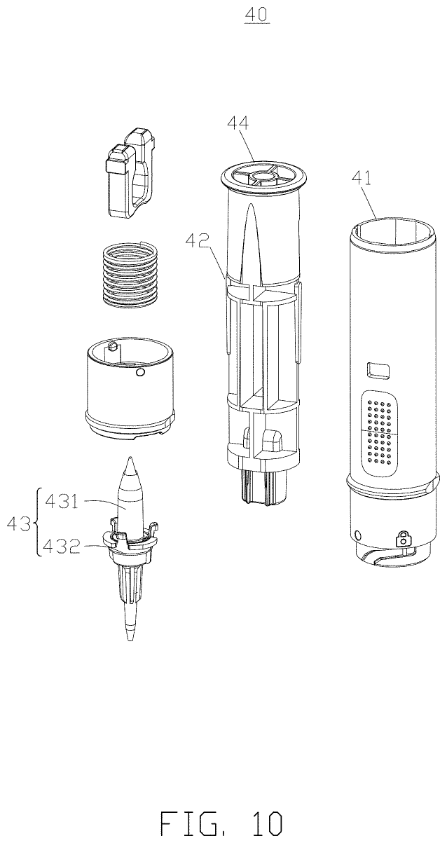

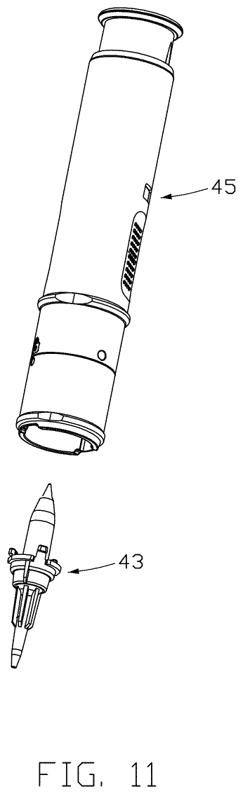

Referring to FIGS. 10 and 11, the liquid transfer unit 40 includes an outer housing 41, an inner housing 42, a liquid extraction assembly 43, and a pressing key 44. The inner housing 42 is clamped with the outer housing 41. The liquid extraction assembly 43 passes through the outer housing 41 and the inner housing 42, and an end of the liquid extraction assembly 43 is extended out of the outer housing 41. The pressing key 44 is disposed on the top of the inner housing 42. In an embodiment, the liquid extraction assembly 43 includes an elastic liquid extraction structure. When in use, the inner housing 42 can be pressed down when the pressing key 44 is pressed, so that the inner housing 42 moves downward along a sidewall of the outer housing 41 to compress the liquid extraction assembly 43. Thus, air inside the elastic liquid extraction structure is extracted out, causing the elastic liquid extraction structure to absorb the detection solution. After absorbing the detection solution, the liquid extraction assembly 43 pushes the inner housing 42 to return to its original position automatically. When the detection solution in the liquid extraction assembly 43 needs to be discharged out, the pressing key 44 can be pressed again to move the inner housing 42 downward relative to the outer housing 41, which further squeezes the liquid extraction assembly 43 to discharge the detection solution. The compression degree of the elastic liquid extraction structure can be controlled to control the volume of the detection solution to achieve quantitative liquid extraction. The liquid transfer unit 40 has the advantages of simple overall structure, low cost, convenient operation, and can achieve the purpose of quantitative.

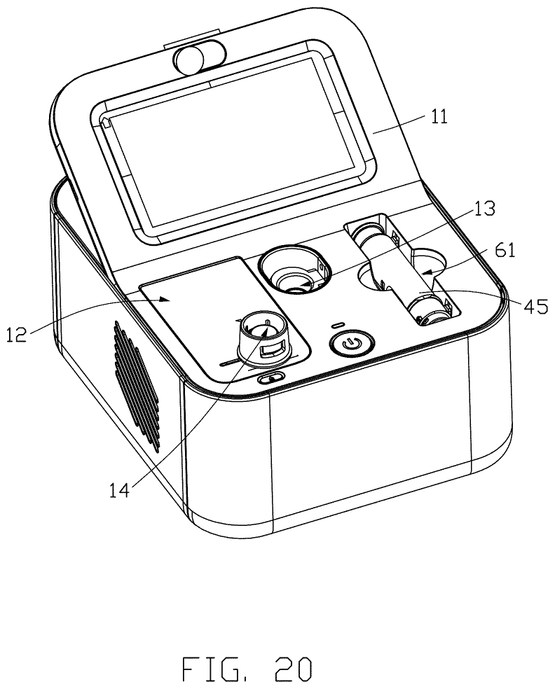

In an embodiment, the outer housing 41, the inner housing 42, and the pressing key 44 constitute a pressing assembly 45, and the liquid extraction assembly 43 cooperates with the pressing assembly 45 to form the liquid transfer unit 40. The liquid extraction assembly 43 is detachably disposed on the pressing assembly 45. The pressing assembly 45 can be used for many times. The liquid extraction assembly 43 is disposable and consumable, and can be replaced at any time to save cost. Therefore, referring to FIGS. 11 and 20, the pressing assembly 45 can be disposed in a pressing assembly mounting area 61 in a nucleic acid detection host 60 to facilitate storage.

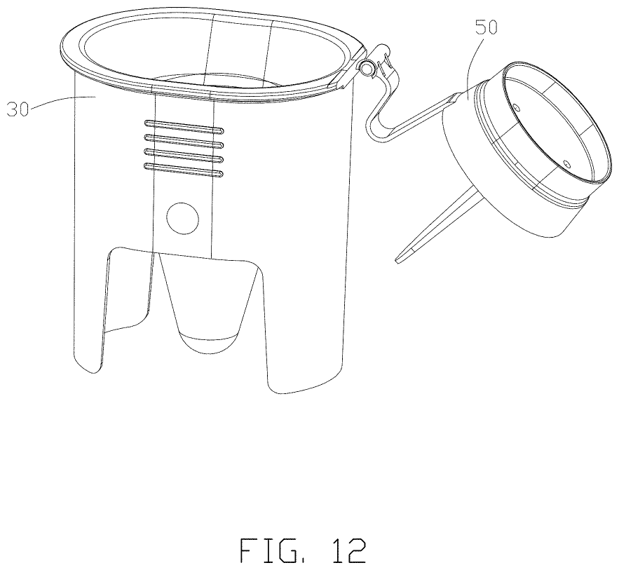

Referring to FIG. 12, the nucleic acid detection device 100 further includes a reagent package 50 for storing a detection reagent (such as a buffer solution). The detection reagent is quantitatively placed in the reagent package 50. The reagent package 50 added into the collection cup 30 can be mixed with a nucleic acid sample to form the detection solution.

In an embodiment, the reagent package 50 is a groove structure with a handle. A detection reagent required for nucleic acid detection is placed in the groove structure, and an opening of the reagent package 50 is sealed by a sealing film. When in use, the user can tear off the sealing film, grasp the handle, pour the detection reagent into the collection cup 30 containing the nucleic acid sample, and then put the collection cup 30 into the holding tank 131 for heating.

In an embodiment, the reagent package 50 is connected to the collection cup 30. Before use, the reagent package 50 is placed in the collection cup 30, which can avoid the loss of the reagent package 50 and can remind the user to add the detection reagent stored in the reagent package 50 into the collection cup 30.

Before the nucleic acid detection, the nucleic acid detection kit 20, the collection cup 30, the liquid transfer unit 40, and the reagent package 50 are packed in a box. The nucleic acid detection kit 20, the collection cup 30, the liquid transfer unit 40, and the reagent package 50 can be provided with an identification code (such as a quick response code and a QR code) to avoid confusion. The identification code can only be set on the collection cup 30 to avoid confusion of the detection solution to be detected.

In an embodiment, the camera 19 is used to record the operation process of the user, and collect the identification code on the collection cup 30.

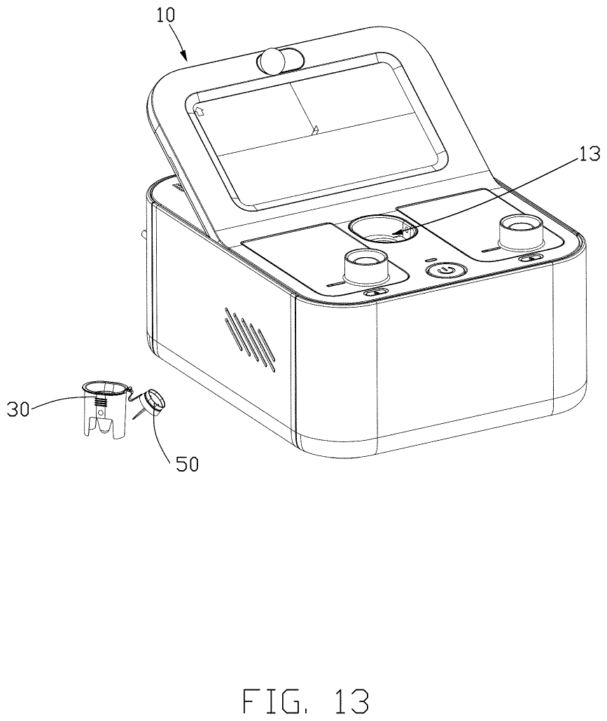

FIGS. 13-17 show steps of a nucleic acid detecting process through the nucleic acid detection device 100 according to an embodiment.

At step one, referring to FIG. 13, operation parameters are set in the nucleic acid detection device 100. The nucleic acid detection host 10 is turned on and the operation parameters are set in the nucleic acid detection host 10.

In an embodiment, the operation parameters include the heating temperature and the heating time of the sample heating area 13, process parameters of the PCR amplification reaction, and process parameters of the electrophoretic detection.

At step two, referring to FIG. 13, the information on the collection cup 30 is collected, and the operation process of the user is recorded.

The camera 19 is turned on to record the operation process of the user. The packaging box containing the nucleic acid detection kit 20, the collection cup 30, and the reagent package 50 is opened. Then the identification code on the collection cup 30 is recorded by the camera 19 to collect relevant information of the nucleic acid sample. The collected information and video data can be uploaded and sent to a client for relevant personnel to view.

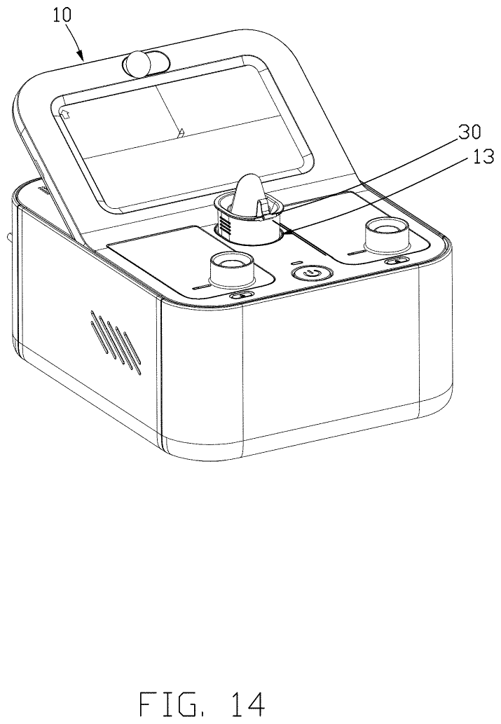

At step three, referring to FIG. 14, the nucleic acid sample is collected by the collection cup 30 to form a detection solution, and the detection solution is heated.

In an embodiment, the nucleic acid sample (such as saliva) is collected by the collection cup 30 and then is heated in the holding tank 131. The heating temperature is in a range from 90° C. to 100° C. and the heating time is in a range from 3 to 8 min. After heating, the saliva is cooled to room temperature or below a preset temperature (such as below 40° C.). After cooling, the detection reagent in the reagent package 50 is added into the collection cup 30 to mix with the saliva to form the detection solution.

In yet another embodiment, the saliva is collected by the collection cup 30. Then the detection reagent in the reagent package 50 is poured into the collection cup 30. The reagent package 50 is buckled at the opening of the collection cup 30. The collection cup 30 is covered and shaken up and down for 3-5 times to obtain the detection solution. Generally, the nucleic acid sample (the saliva) and the detection reagent can be mixed evenly by shaking the collection cup 30 up and down for 5 times. The collection cup 30 containing the detection solution is inserted into the holding tank 131. When the collection cup 30 is inserted into the holding tank 131, the second sensor sends a trigger signal to the controller 16 to initiate the heating process. The heating temperature is in a range from 90° C. to 100° C., and the heating time is in a range from 3 to 8 min. Then the holding tank 131 is cooled to room temperature or below a preset temperature (such as below 40° C.). In an embodiment, a temperature sensor and a time relay are used to sense the heating temperature and the heating time.

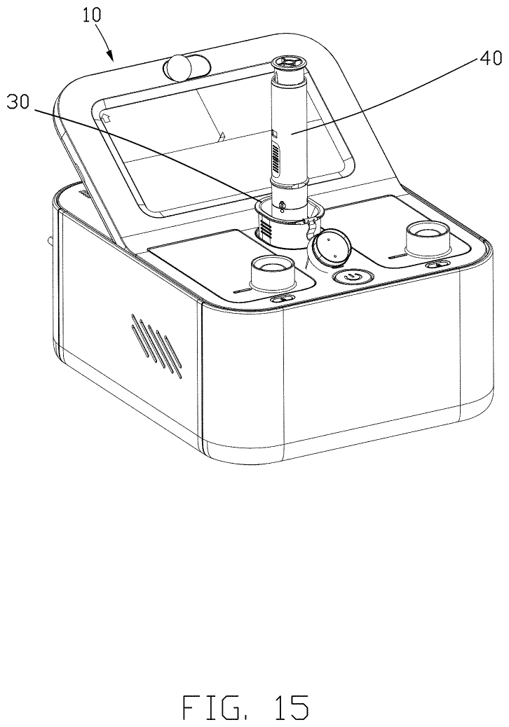

At step four, referring to FIG. 15, the nucleic acid detection kit 20 is inserted into the mounting groove 121. The first sensor senses the insertion of the nucleic acid detection kit 20, and then automatically starts the nucleic acid detection.

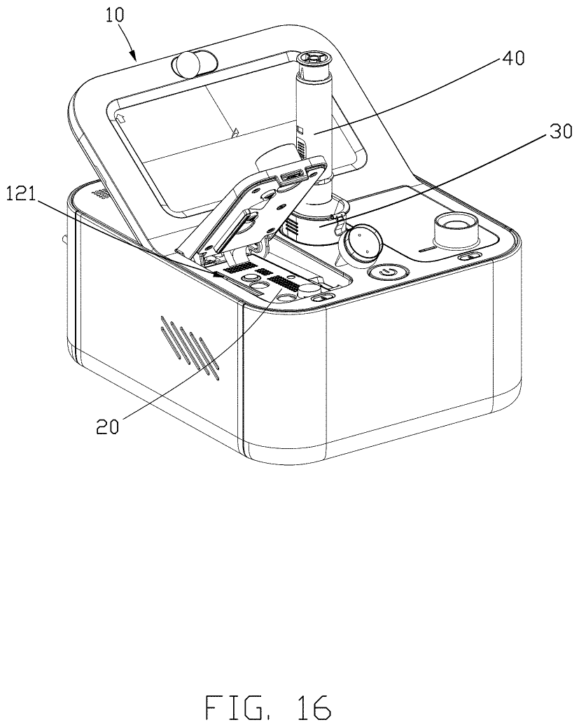

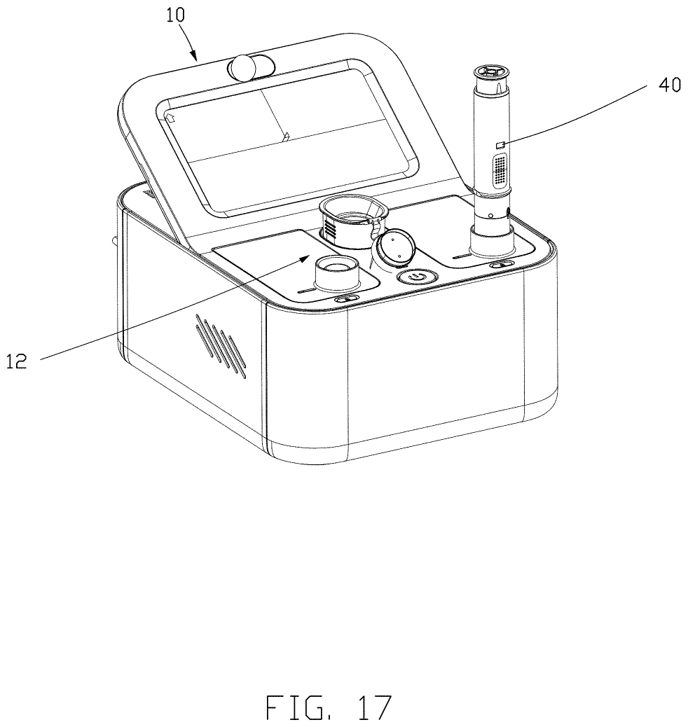

At step five, referring to FIGS. 16 and 17, the detection solution is transferred from the collection cup 30 into the nucleic acid detection kit 20 to perform the PCR amplification reaction and the electrophoretic detection.

In an embodiment, the detection solution is quantitatively sucked 10-30 μl (preferably 20 μl) by the liquid transfer unit 40 from the collection cup 30 and is added into the nucleic acid detection kit 20. The detection solution containing the nucleic acid sample is undergone the PCR amplification reaction in the detection chip 23. After amplification, the detection solution is combined with a fluorescent reagent received in the detection chip 23 to form a product with fluorescent groups. Then the product with fluorescent groups enters the electrophoretic box 24 from the detection chip 23 to undergo the electrophoretic detection.

At step six, an electrophoretic detection result (such as the fluorescent image) is acquired by the image collection unit 15.

After the electrophoretic detection, the fluorescent image is acquired by the image collection unit 15. The fluorescent image is processed by the image processor, and then displayed on the display screen 18. The fluorescent image can also be uploaded and sent to the client for the user to consult.

At step seven, the nucleic acid detection is over.

After the nucleic acid detection, the collection cup 30, the liquid transfer unit 40, and the nucleic acid detection kit 20 are removed from the nucleic acid detection device 100 and put into the packaging box for recycling.

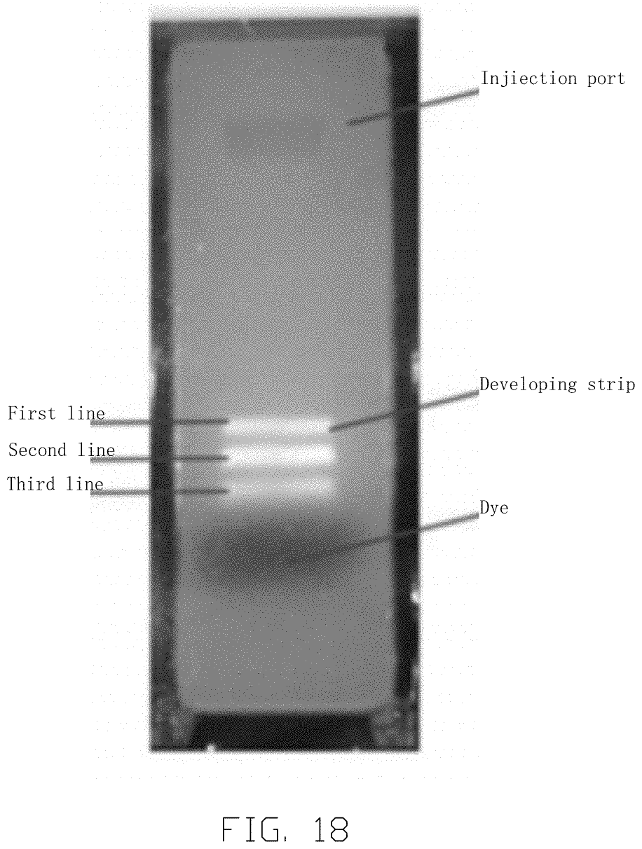

A fluorescent image of a nucleic acid detection result obtained by using the nucleic acid detection device 100 is shown in FIG. 18. In this embodiment, by predefining a range of each line on a standard fluorescent image, the nucleic acid detection device 100 can automatically identify the nucleic acid detection result when the fluorescent image is obtained. If a labeling position of a first line on the fluorescent image is within a predefined range, it can be determined that human genes are included in the nucleic acid sample. If the labeling position of the first line is not within the predefined range, it can be determined that human genes are not included in the nucleic acid sample. If a labeling position of a second line on the fluorescent image is within the predefined range, it can be determined that RNA replicase is included in the nucleic acid sample. If the labeling position of the second line is not within the predefined range, it can be determined that RNA replicase is not included in the nucleic acid sample. If a labeling position of a third line on the fluorescent image is within the predefined range, it can be determined that the nucleic acid sample includes N protein. If the labeling position of the third line is not within the predefined range, it can be determined that the nucleic acid sample does not include N protein.

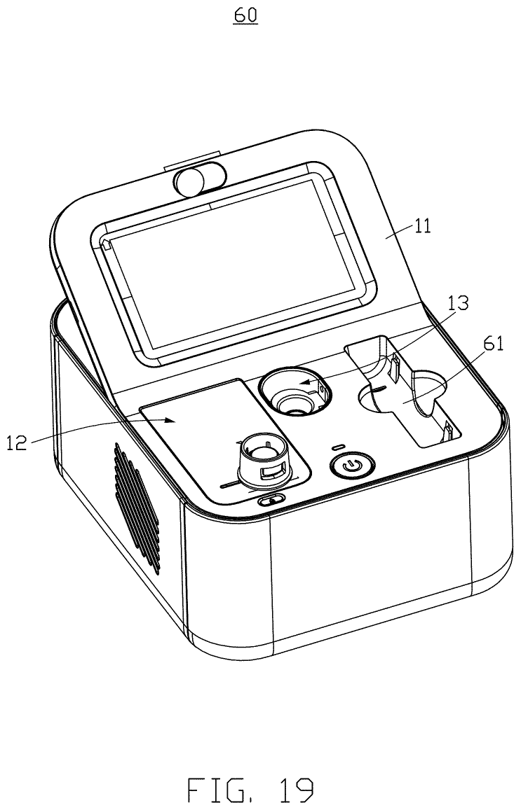

FIGS. 19 and 20 illustrate yet another nucleic acid detection host 60 according to the present disclosure. The nucleic acid detection host 60 includes only one detection kit installation area 12. At the same time, the nucleic acid detection host 60 further includes the pressing assembly mounting area 61 for storing the pressing assembly 45. It can be understood that the pressing assembly mounting area 61 can also be designed as other functional areas to make full use of the space of the nucleic acid detection host 60.

The nucleic acid detection device 100 provided by the present disclosure can integrate the PCR amplification reaction and the electrophoretic detection of nucleic acid into a single equipment through the cooperation of the nucleic acid detection host 10 and the nucleic acid detection kit 20. Thus, the nucleic acid detection device 100 has a simple structure, which is portable, flexible, and convenient, and can be used at home. At the same time, the detecting process is flexible, which does not need to be carried out in a professional laboratory.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure, up to and including, the full extent established by the broad general meaning of the terms used in the claims.

What is claimed is:

1. A nucleic acid detection host, comprising:
a host body;
a detection kit installation area disposed on the host body;
a sample heating area disposed on the host body;
a sampling groove;
a heating structure disposed in the detection kit installation area; and
an image collection unit;
wherein the detection kit installation area comprises a mounting groove and a cover plate detachably disposed on the mounting groove, the mounting groove is configured to detachably install a nucleic acid detection kit therein, the sample heating area is configured to collect a detection solution and heat the detection solution, the sampling groove is disposed on the cover plate and connected to the mounting groove, the sampling groove is configured to add the detection solution into the nucleic acid detection kit installed in the mounting groove, the heating structure is configured to heat the nucleic acid detection kit to perform a PCR amplification reaction and an electrophoretic detection, the image collection unit is disposed on a side of the mounting groove away from the sampling groove, and is configured to collect an image of the nucleic acid detection kit.

2. The nucleic acid detection host of claim 1, wherein the heating structure comprises a first heating component disposed in the mounting groove and a second heating component disposed on a surface of the cover plate close to the mounting groove.

3. The nucleic acid detection host of claim 2, wherein the first heating component comprises a first circuit board and a plurality of first heaters disposed on the first circuit board, the first circuit board is disposed on a side of the mounting groove away from the cover plate, each of the plurality of first heaters extends in the mounting groove and connects to the nucleic acid detection kit in the mounting groove.

4. The nucleic acid detection host of claim 2, wherein the second heating component comprises a second circuit board and a plurality of second heaters disposed on the second circuit board, the second circuit board is disposed inside the cover plate, each of the plurality of second heaters protrudes from the surface of the cover plate close to the mounting groove to connect to the nucleic acid detection kit in the mounting groove.

5. The nucleic acid detection host of claim 1, wherein the detection kit installation area further comprises an imaging port disposed on a bottom surface of the mounting groove and a host connector disposed on a surface of the cover plate close to the mounting groove, the host connector is electrically connected to the nucleic acid detection kit in the mounting groove, the image collection unit is disposed on a side of the imaging port away from the sampling groove, the image collection unit is configured to collect a fluorescent image in the nucleic acid detection kit through the imaging port.

6. The nucleic acid detection host of claim 1, wherein relative to a first surface of the host body, a height of an end of the mounting groove closed to the sampling groove is lower than a height of another end of the mounting groove away from the sampling groove.

7. The nucleic acid detection host of claim 1, wherein the sample heating area comprises a holding tank and a heating block disposed at a bottom of the holding tank.

8. The nucleic acid detection host of claim 1, further comprising a first sensor disposed on the detection kit installation area and a second sensor disposed on the sample heating area, wherein the first sensor is disposed to sense whether the nucleic acid detection kit is inserted into the detection kit installation area, and the second sensor is configured to sense whether the detection solution is added into the sample heating area.

9. The nucleic acid detection host of claim 1, further comprising a display screen and a camera, wherein the display screen is configured to display the image of the nucleic acid detection kit, and the camera is configured to record an identification information of the detection solution.

10. The nucleic acid detection host of claim 1, wherein the nucleic acid detection host comprises two detection kit installation areas.

11. The nucleic acid detection host of claim 10, wherein an isolation layer is disposed between the two detection kit installation areas.

12. The nucleic acid detection host of claim 1, further comprising a pressing assembly storage area, wherein the pressing assembly mounting area is configured to store a liquid transfer unit.

13. A nucleic acid detection device, comprising:
a nucleic acid detection host, comprising:
a host body;
a detection kit installation area disposed on the host body;
a sample heating area disposed on the host body;
a sampling groove;
a heating structure disposed in the detection kit installation area; and
an image collection unit;
wherein, the detection kit installation area comprises a mounting groove and a cover plate detachably disposed on the mounting groove, the mounting groove is configured to detachably install a nucleic acid detection kit therein, the sample heating area is configured to collect a detection solution and heat the detection solution, the sampling groove is disposed on the cover plate and is connected to the mounting groove, the sampling groove is configured to add the detection solution into the nucleic acid detection kit installed in the mounting groove, the heating structure is configured to heat the nucleic acid detection kit to perform a PCR amplification reaction and an electrophoretic detection, the image collection unit is disposed on a side of the mounting groove away from the sampling groove, and is configured to collect an image of the nucleic acid detection kit;
a collection cup;
a liquid transfer unit; and
the nucleic acid detection kit,
wherein the collection cup is detachably disposed in the sample heating area, the collection cup is configured to contain the detection solution, the liquid transfer unit is detachably disposed in the sample heating area or the collection cup, the liquid transfer unit is configured to quantitatively transfer the detection solution from the collection cup into the nucleic acid detection kit, the nucleic acid detection kit is configured to perform the PCR amplification reaction and the electrophoretic detection.

14. The nucleic acid detection device of claim 13, wherein the heating structure comprises a first heating component disposed in the mounting groove and a second heating component disposed on a surface of the cover plate close to the mounting groove.

15. The nucleic acid detection device of claim 14, wherein the first heating component comprises a first circuit board and a plurality of first heaters disposed on the first circuit board, the first circuit board is disposed on a side of the mounting groove away from the cover plate, each of the plurality of first heaters extends in the mounting groove and connects to the nucleic acid detection kit in the mounting groove.

16. The nucleic acid detection device of claim 14, wherein the second heating component comprises a second circuit board and a plurality of second heaters disposed on the second circuit board, the second circuit board is disposed inside the cover plate, each of the plurality of second heaters protrudes from the surface of the cover plate close to the mounting groove to connect to with the nucleic acid detection kit in the mounting groove.

17. The nucleic acid detection device of claim 13, wherein the nucleic acid detection host comprises two detection kit installation areas.

18. The nucleic acid detection device of claim 17, wherein an isolation layer is disposed between the two detection kit installation areas.

19. The nucleic acid detection device of claim 13, further comprising a pressing assembly storage area, wherein the pressing assembly storage area is configured to store a liquid transfer unit.

20. The nucleic acid detection device of claim 13, wherein the nucleic acid detection kit comprises:
a kit body;
a sampling port disposed on the kit body;
a detection chip disposed in the kit body;
an electrophoretic box disposed outside the kit body; and
a detection kit connector disposed in the kit body,
wherein the detection chip is connected to the sampling groove through the sampling port, the detection chip is connected to the electrophoretic box, the detection kit connector is electrically connected to the detection chip and the electrophoretic box.
